(19) United States
(12) Patent Application Publication
(10) Pub. No.: US 2020/0030471 A1
(43) Pub. Date: Jan. 30, 2020

(54) STEAM CABINET

(71) Applicants: THE BOARD OF TRUSTEES OF THE UNIVERSITY OF ALABAMA, Tuscaloosa, AL (US); NOVTECH LLC, Chattanooga, TN (US)

(72) Inventors: Stanley Weller, Marietta, GA (US); Lloyd L. Starks, Spring City, TN (US); David William Macphee, Tuscaloosa, AL (US); Kellis Christian Kincaid, Tuscaloosa, AL (US); Steven Ryley Pitts, Tuscaloosa, AL (US); Nicholas Corey Larsen, Tuscaloosa, AL (US); Jonathan George Cappola, Tuscaloosa, AL (US); Tim A. Haskew, Northport, AL (US)

(21) Appl. No.: 16/526,615

(22) Filed: Jul. 30, 2019

Related U.S. Application Data
(60) Provisional application No. 62/711,953, filed on Jul. 30, 2018.

(51) Int. Cl. A61L 2/07; A61L 2/26; B08B 3/08

(52) U.S. Cl. CPC A61L 2/07; A61L 2/26; B08B 2230/01; A61L 2202/15; A61L 2202/17; B08B 3/08

(57) ABSTRACT

Various implementations include a steam cabinet. The steam cabinet includes at least one chamber wall, a door, at least one heating element, at least one steam generator, a supply fan, and an exhaust fan. The at least one chamber wall defines a processing chamber. The door is for allowing access to the processing chamber. The at least one heating element is configured to provide heat to the processing chamber. The at least one steam generator is configured to provide steam to the processing chamber. The supply fan has a supply fan inlet and a supply fan outlet. The supply fan outlet is in fluid communication with the at least one heating element and the processing chamber. The exhaust fan has an exhaust fan inlet and an exhaust fan outlet. The exhaust fan inlet is in fluid communication with the processing chamber.

### BACKGROUND

Sports equipment, such as pads, helmets, and shoes regularly get soiled from dirt and bodily fluids after use. Similarly, equipment from police and fire departments, hospitals and medical devices, veterinary equipment, and military equipment, to name a few, often get soiled after use. Cleaning, disinfecting, sanitizing, and/or deodorizing is thus essential to maintaining hygiene. However, such equipment is often bulky and thus not amenable to washing machines. Moreover, the quantity of equipment that must be treated is often very large, making the individual treatment of equipment time consuming. Thus, a need exists for a device to clean, disinfect, sanitize, and/or deodorize equipment thoroughly and easily. A need also exists for a device that can accomplish this with multiple sets of equipment. The devices and methods disclosed herein address these and other needs.

### SUMMARY

Various implementations include a steam cabinet. The steam cabinet includes at least one chamber wall, a door, at least one heating element, at least one steam generator, a supply fan, and an exhaust fan. The at least one chamber wall defines a processing chamber. The door is for allowing access to the processing chamber. The at least one heating element is configured to provide heat to the processing chamber. The at least one steam generator is configured to provide steam to the processing chamber. The supply fan has a supply fan inlet and a supply fan outlet. The supply fan outlet is in fluid communication with the at least one heating element and the processing chamber. The exhaust fan has an exhaust fan inlet and an exhaust fan outlet. The exhaust fan inlet is in fluid communication with the processing chamber.

In some implementations, the at least one chamber wall includes a first chamber wall, a second chamber wall, a third chamber wall, a fourth chamber wall, a bottom chamber wall, and a top chamber wall. The first chamber wall defines a door opening, and the door is configured to seal the door opening when the door is in a closed position.

In some implementations, the at least one chamber wall includes insulation.

In some implementations, a bottom portion of the fourth chamber wall defines a steam injection port for providing steam from the at least one steam generator to the processing chamber.

In some implementations, the steam cabinet includes an air filter in fluid communication with the supply fan outlet. In some implementations, the air filter is a HEPA air filter.

In some implementations, the at least one steam generator is configured to deliver an agent for cleaning, disinfecting, sanitizing, deodorizing, or any combination thereof. In some implementations, the agent includes an antimicrobial agent.

In some implementations, the at least one heating element and the at least one steam generator are configured to increase a temperature of the processing chamber to 167 degrees F. or more.

In some implementations, the door includes a magnetic locking mechanism.

In some implementations, the steam cabinet includes a temperature sensor disposed within the processing chamber.

Various other implementations include a method of using a steam cabinet. The method includes providing a steam cabinet that includes at least one chamber wall, a door, at least one heating element, at least one steam generator, a supply fan, and an exhaust fan. The at least one chamber wall defines a processing chamber. The door is for allowing access to the processing chamber. The at least one heating element is configured to provide heat to the processing chamber. The at least one steam generator is configured to provide steam to the processing chamber. The supply fan has a supply fan inlet and a supply fan outlet. The supply fan outlet is in fluid communication with the at least one heating element and the processing chamber. The exhaust fan has an exhaust fan inlet and an exhaust fan outlet. The exhaust fan inlet is in fluid communication with the processing chamber. The method also includes disposing an item to be steamed within the processing chamber, activating the at least one heating element, the supply fan, and the at least one steam generator to increase a temperature of the processing chamber to 167 degrees F. or more for a predetermined period of time, and activating the exhaust fan to remove excess steam from the processing chamber.

In some implementations, the at least one chamber wall includes a first chamber wall, a second chamber wall, a third chamber wall, a fourth chamber wall, a bottom chamber wall, and a top chamber wall. The first chamber wall defines a door opening, and the door is configured to seal the door opening when the door is in a closed position.

In some implementations, the at least one chamber wall includes insulation.

In some implementations, a bottom portion of the fourth chamber wall defines a steam injection port for providing steam from the at least one steam generator to the processing chamber.

In some implementations, the steam cabinet includes an air filter in fluid communication with the supply fan outlet. In some implementations, the air filter is a HEPA air filter.

In some implementations, the at least one steam generator is configured to deliver an agent for cleaning, disinfecting, sanitizing, deodorizing, or any combination thereof. In some implementations, the agent includes an antimicrobial agent.

In some implementations, the door includes a magnetic locking mechanism.

In some implementations, the steam cabinet includes a temperature sensor disposed within the processing chamber.

### BRIEF DESCRIPTION OF DRAWINGS

Example features and implementations are disclosed in the accompanying drawings. However, the present disclosure is not limited to the precise arrangements and instrumentalities shown.

FIG. 1 is a perspective view of a steam cabinet with door closed, according to one implementation.

FIG. 2 is a perspective view of the steam cabinet of FIG. 1 with door open.

FIG. 3 is a perspective view of the secondary chamber of the steam cabinet of FIG. 1 with the supply fan and exhaust fan visible.

FIG. 4 is a perspective view of a VaporJet 8000 Steam Generator on the steam cabinet of FIG. 1.

FIG. 5 is a perspective view of a magnetic locking mechanism of a door on the steam cabinet of FIG. 1.

FIG. 6 is a perspective view of the control panel of the steam cabinet of FIG. 1.

FIG. 7 is a perspective view of a large equipment rack that can be used in the steam cabinet of FIG. 1.

FIG. 8 is a perspective view of a small equipment rack that can be used in the steam cabinet of FIG. 1.

FIG. 9 is a perspective view of exhaust fan duct work extending from the back of the steam cabinet of FIG. 1.

FIG. 10 is a perspective view of the air filter of the steam cabinet of FIG. 1.

FIG. 11 is a perspective view of a control chassis mounted to the back of the steam cabinet of FIG. 1.

FIG. 12 is a perspective view of an interior of a control chassis of the steam cabinet of FIG. 1. Arduino is pictured at top left, power relays are at top right, and 12 VDC power supply is at left center.

FIG. 13 is a schematic of an example relay for 220 VAC component.

FIG. 14 is a perspective view of the steam cabinet of FIG. 1 set up for an experiment with a large equipment rack and equipment.

FIG. 15 is a perspective view of the steam cabinet of FIG. 1 set up for an experiment with a small equipment rack and equipment.

FIG. 16 is a schematic for the steam cabinet of FIG. 1.

FIG. 17A is a graph showing change in mass versus steam injection duration (fixed pressure and oven temperature).

FIG. 17B is a graph showing change in mass versus steam pressure (fixed injection duration and oven temperature).

FIG. 17C is a graph showing change in mass versus oven temperature (fixed steam pressure and injection duration).

FIG. 17D is a graph showing change in mass versus vacuum pressure (fixed injection duration and oven temperature).

FIG. 18A is a graph showing steam cabinet heating starting from a cold cabinet with no load, of a steam cabinet disclosed herein.

FIG. 18B is a graph showing steam cabinet heating starting from a cold cabinet with a full load, of a steam cabinet disclosed herein.

FIG. 18C is a graph showing steam cabinet heating starting from a warm cabinet with a full load, of a steam cabinet disclosed herein.

FIG. 18D is a graph showing steam cabinet heating starting from a cold cabinet with a half-sized load, of a steam cabinet disclosed herein.

FIG. 18E is a graph showing steam cabinet heating starting from a warm cabinet with a half-sized load, of a steam cabinet disclosed herein.

### DETAILED DESCRIPTION

Various implementations include a steam cabinet. The steam cabinet includes at least one chamber wall, a door, at least one heating element, at least one steam generator, a supply fan, and an exhaust fan. The at least one chamber wall defines a processing chamber. The door is for allowing access to the processing chamber. The at least one heating element is configured to provide heat to the processing chamber. The at least one steam generator is configured to provide steam to the processing chamber. The supply fan has a supply fan inlet and a supply fan outlet. The supply fan outlet is in fluid communication with the at least one heating element and the processing chamber. The exhaust fan has an exhaust fan inlet and an exhaust fan outlet. The exhaust fan inlet is in fluid communication with the processing chamber.

Various other implementations include a method of using a steam cabinet. The method includes providing a steam cabinet that includes at least one chamber wall, a door, at least one heating element, at least one steam generator, a supply fan, and an exhaust fan. The at least one chamber wall defines a processing chamber. The door is for allowing access to the processing chamber. The at least one heating element is configured to provide heat to the processing chamber. The at least one steam generator is configured to provide steam to the processing chamber. The supply fan has a supply fan inlet and a supply fan outlet. The supply fan outlet is in fluid communication with the at least one heating element and the processing chamber. The exhaust fan has an exhaust fan inlet and an exhaust fan outlet. The exhaust fan inlet is in fluid communication with the processing chamber. The method also includes disposing an item to be steamed within the processing chamber, activating the at least one heating element, the supply fan, and the at least one steam generator to increase a temperature of the processing chamber to 167 degrees F. or more for a predetermined period of time, and activating the exhaust fan to remove excess steam from the processing chamber.

Disclosed herein is a steam cabinet 100 into which items, such as equipment, can be loaded and batch-processed. The disclosed steam cabinet 100 and methods for using the steam cabinet 100 utilize electric and steam heating and can include an injected Steam-N-Shield disinfectant to disinfect equipment.

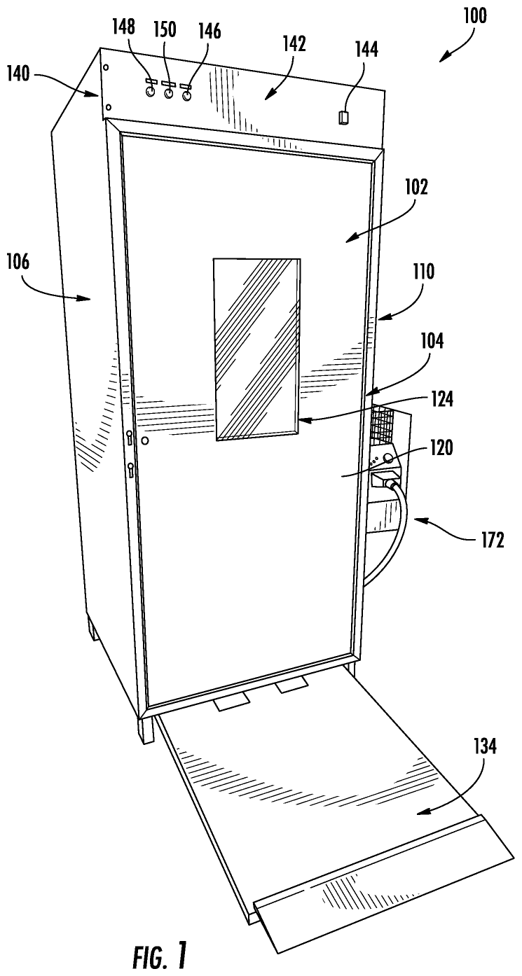

FIG. 1 shows a steam cabinet 100 having a processing chamber 102, a secondary chamber 140, a heating element 160, a steam generator 172, a supply fan 162, an exhaust fan 180, and a controller 192.

The processing chamber 102 is the portion of the steam cabinet 100 where items are disposed for being steamed. The processing chamber 102 is defined by a first chamber wall 104, a second chamber wall 106, a third chamber wall 108, a fourth chamber wall 110, a bottom chamber wall 112, and a top chamber wall 114. Each of the walls 104, 106, 108, 110, 112, 114 include insulation 116 to prevent the loss of heat during use and includes stainless steel to prevent corrosion. Insulation 116 can make the steam cabinet 100 outside surfaces cool to the touch and the initial heat-up time for the steam cabinet 100 can be reduced. The processing chamber 102 shown in FIG. 1 is of sufficient size to fit a large equipment rack.

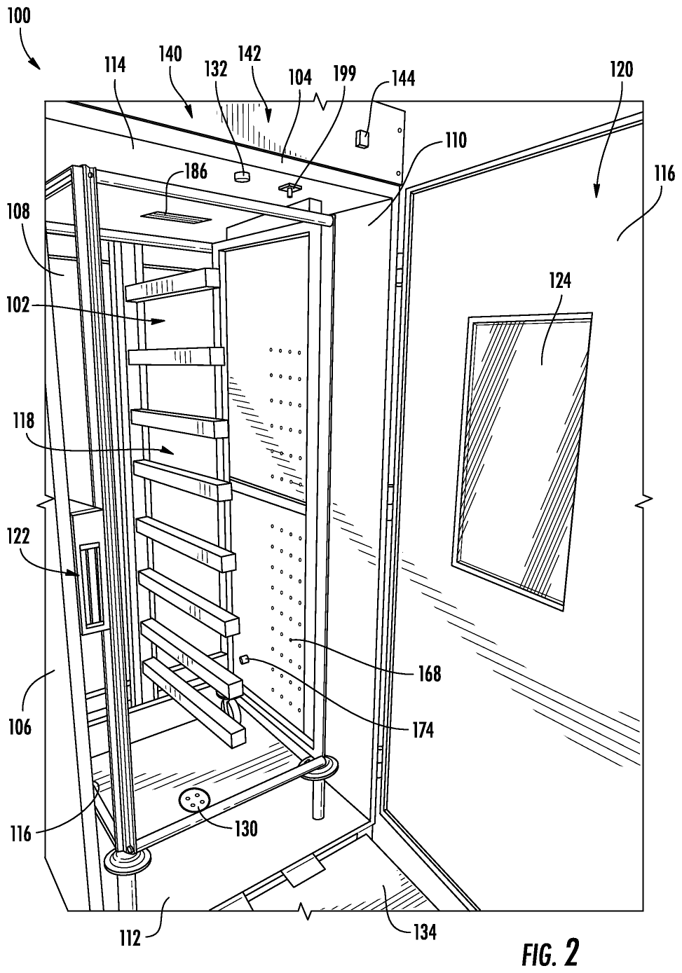

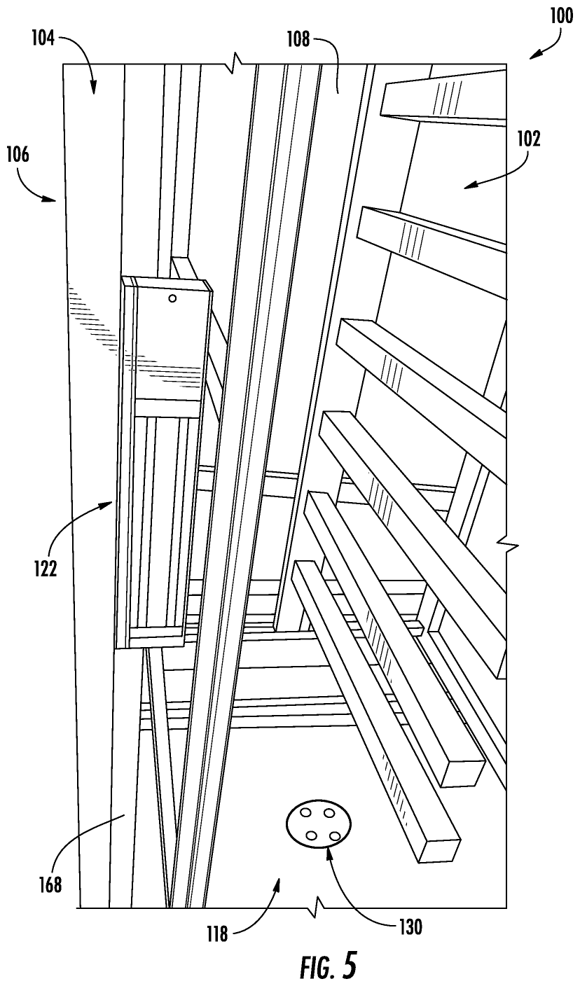

The first chamber wall 104 defines a door opening 118. A door 120 is hingedly coupled to the first chamber wall 104 and is rotatable between an open position (shown in FIG. 2) for loading items into the processing chamber 102 and a closed position (shown in FIG. 1) wherein the door 120 creates an airtight seal between the door 120 and the first chamber wall 104. The door 120 includes a magnetic locking device 122, as shown in FIG. 5. When the door 120 is in the closed position, the magnetic locking device 122 can be activated to lock the door 120 in the closed position such that the door 120 cannot be rotated to the open position. The magnetic locking mechanism 122 is included in the steam cabinet 100 to ensure safety of the operator. The magnetic locking mechanism 122 prevents the operator from opening the steam cabinet 100 during the batch process, protecting the operator from accidental exposure to hot steam. The magnetic locking mechanism 122 can be activated while the steam cabinet 100 is powered on but can be unlocked with the press of a button on the control panel 142. Like the chamber walls 104, 106, 108, 110, 112, 114, the door 120 also includes insulation 116. A window 124 is also included in the door 120 for viewing the processing chamber 102 during use.

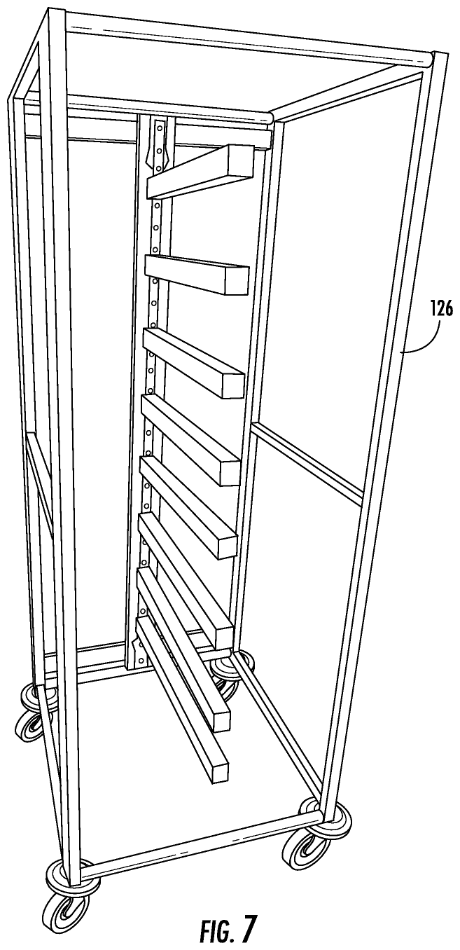

FIGS. 7 and 8 show two equipment racks 126, 128 that can be disposed within the processing chamber 102 for holding items to be steamed. The first rack 126, shown in FIG. 7, is used for hanging up to eight sets of larger pads (shoulder, rib, etc.) simultaneously. Each branch on the first rack 126 is supported by a metal bracket. The second rack 128, shown in FIG. 8, is used for holding smaller pads (elbow, knee, etc.) and equipment such as shoes and helmets. The grates of the second rack 128 can be adjusted vertically to meet the needs of the operator. Other racks can also be used, e.g., those with hooks or hangers for jersey and pants.

FIG. 2 also shows a drain 130 disposed in the bottom chamber wall 112 of the processing chamber 102. After running the sanitization cycle of the steam cabinet 100, a small amount of condensation may form on the bottom chamber wall 112 of the steam cabinet 100. The drain 130 allows for the condensation to drain from the processing chamber 102 after use. Furthermore, the drain 130 can be used when the steam cabinet 100 is intermittently cleaned to combat the build-up of foreign material and residue from cleaning agents found in the small amount of condensation.

The processing chamber 102 further includes an emergency stop button 132 installed on the top chamber wall 114 inside the processing chamber 102, as shown in FIG. 2. In the event an operator becomes trapped inside the steam cabinet 100, the emergency stop button 132 can be pressed to immediately power down the steam cabinet 100. This interrupts the steam generator 172, heating elements 160, and supply fan 162, and releases the magnetic locking mechanism 122 of the door 120.

A ramp 134 is shown in FIG. 1. The ramp 134 allows the operator to easily load racks into the processing chamber 102. The ramp 134 in FIG. 1 is constructed of ¾″ plywood with aluminum trim affixed to the end, but in other implementations, the ramp can be made of any material. The ramp 134 is attached to the base of the steam cabinet 100 with metal hinges and can be easily folded inside of the processing chamber 102 when the steam cabinet 100 is not in use or is being transported.

To make the steam cabinet 100 movable with a standard forklift, and to allow for installation of a drainage system if desired, a structure can be fabricated to raise the steam cabinet 100 off the ground, as shown in FIG. 1.

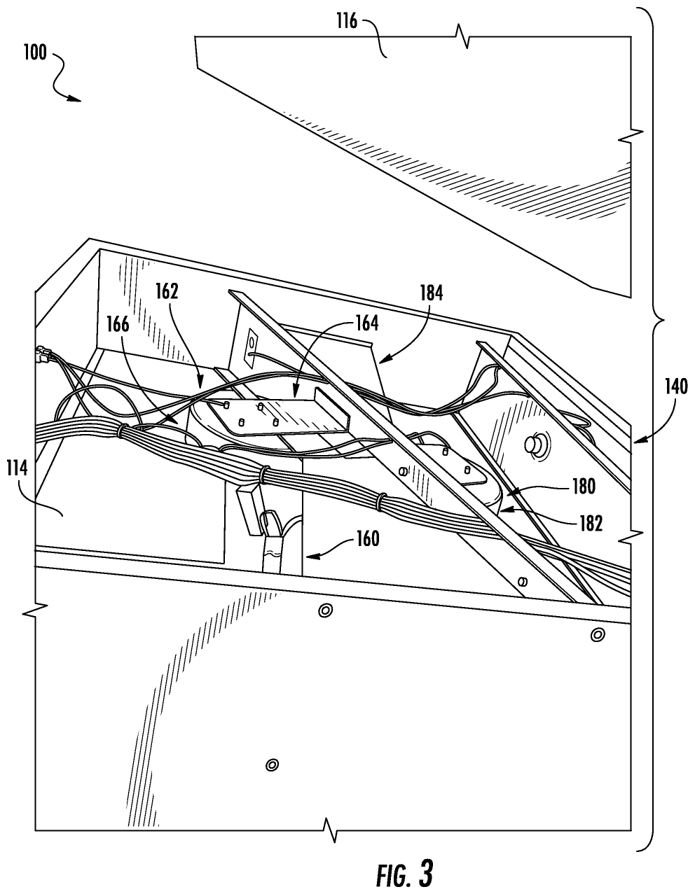

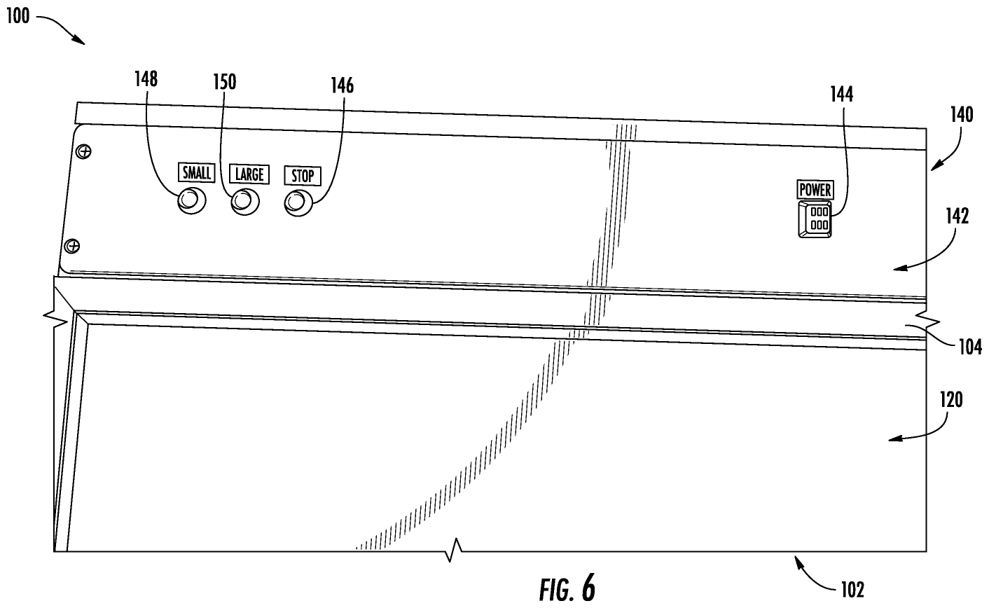

The secondary chamber 140 is disposed above the top chamber wall 114 of the processing chamber 102, as shown in FIG. 3. The heating element 160, supply fan 162, and exhaust fan 180 are disposed within the secondary chamber 140. The secondary chamber 140 also includes a control panel 142, as shown in FIG. 6. The control panel 142 includes a power switch 144 for supplying power to the steam cabinet 100, a stop button 146, a “small” button 148 for steaming small batches of items, and “large” button 148 for steaming large batches of items. The switches and buttons of the control panel 142 are discussed in further detail below with reference to the controller 192.

In some implementations, the control panel of the steam cabinet can contain a switch to power the cabinet on, and a rotating temperature dial to adjust the heat produced. In a preferred implementation, the steam cabinet is always set to provide a maximum temperature inside the processing chamber, but in other implementations, the control panel can include a temperature dial to vary the temperature of the processing chamber. In some implementations, a dial is included to select for different equipment sizes. In some implementations, the steam cabinet includes a gas-expansion mechanical thermostat and with a control knob. In some implementations, the steam cabinet can include a temperature sensor with a display of the temperature reading on the control panel. In some implementations, the steam cabinet includes a combistat for controlling the pressure and temperature of the steam. In some implementations any of the devices disposed within the secondary chamber shown in FIG. 3 can be disposed anywhere else on the steam cabinet based on space constraints and efficiency.

The steam cabinet 100 shown in FIGS. 1-15 is 34 inches deep, 34 inches wide, and 77 inches in height. However, in other implementations, the dimensions can vary depending on the particular location of use, customer desires, quantity and size of equipment to be used, and the like. In some implementations, the depth can vary from 12 inches to 96 inches, the width can vary from 12 inches to 96 inches, and the height can vary from 12 inches to 96 inches. In some implementations, the depth, width, and height can be any size to accommodate various item and batch sizes.

The heating element 160 is shown in the secondary chamber 140 in FIG. 3. The heating element 160 provides heat to the processing chamber 102. The heating element 160 is in fluid communication with the supply fan 162, which is used for delivering the heat from the heating element 160 to the processing chamber 102. The supply fan 162 has a supply fan inlet 164 and a supply fan outlet 166. The supply fan outlet 166 is ducted to and in fluid communication with the processing chamber 102, and the supply fan inlet 164 is ducted to and in fluid communication with the outside of the steam cabinet 100. Air ports 168 are located along the length of the second chamber wall 106 and fourth chamber wall 110 of the processing chamber 102 to allow the air flowing from the supply fan inlet 164 to enter the processing chamber 102, as shown in FIG. 2. The heating element 160 shown in FIG. 3 includes two 220 VAC, 1 k W heating elements. The supply fan 162 shown in FIG. 3 includes two 150 W fans for moving air across the heating element 160 and into the processing chamber 102. In some implementations, the heating element includes a 220 VAC 2.5 kW heating element.

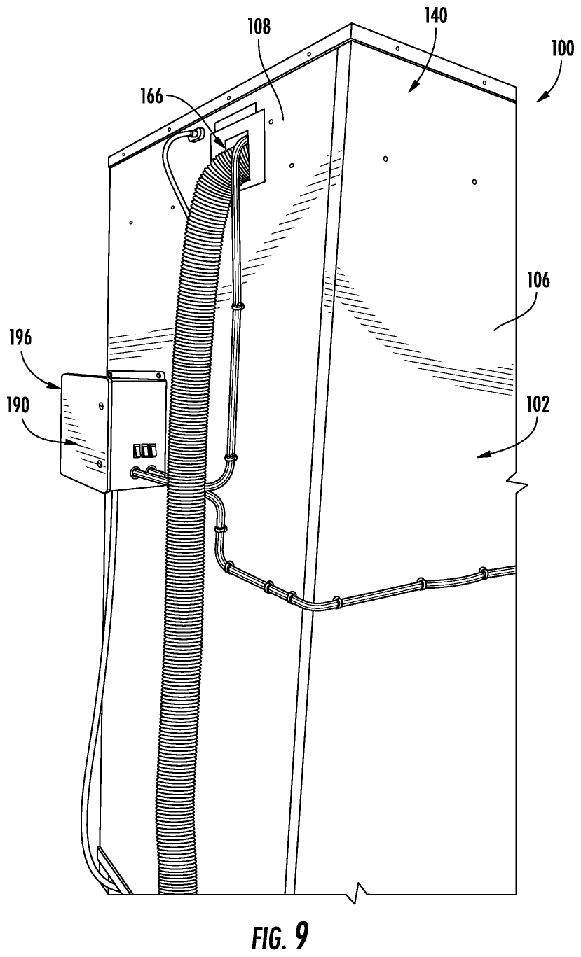

FIG. 10 shows a HEPA air filter 170 included at the supply fan inlet 164 to ensure that air drawn into the supply fan 162 is sanitary. The HEPA air filter 170 prevents air from an unclean (e.g., hospital) environment from entering the processing chamber 102 in situations where sterilization is important. In other implementations, the air filter can be any quality of air filter suitable for the application. In some implementations, no air filter is included in the steam cabinet.

The steam generator 172 is configured to provide steam to the processing chamber 102. The steam generator 172 is ducted to a steam injection port 174 that is defined by a bottom portion of the fourth chamber wall 112. Because the steam injection port 174 is toward the bottom of the steam cabinet 100, the distance from the steam injection port 174 to the steam generator 172 is minimize. Consequently, the steam injection port 174 being close to the base of the steam cabinet 100 allows for the steam to naturally rise, permeating the processing chamber 102. The short distance from the steam generator 172 also minimizes the formation of undesired condensation as the steam travels from the steam generator 172 into the processing chamber 102. This placement can provide high quality steam inside the processing chamber 102.

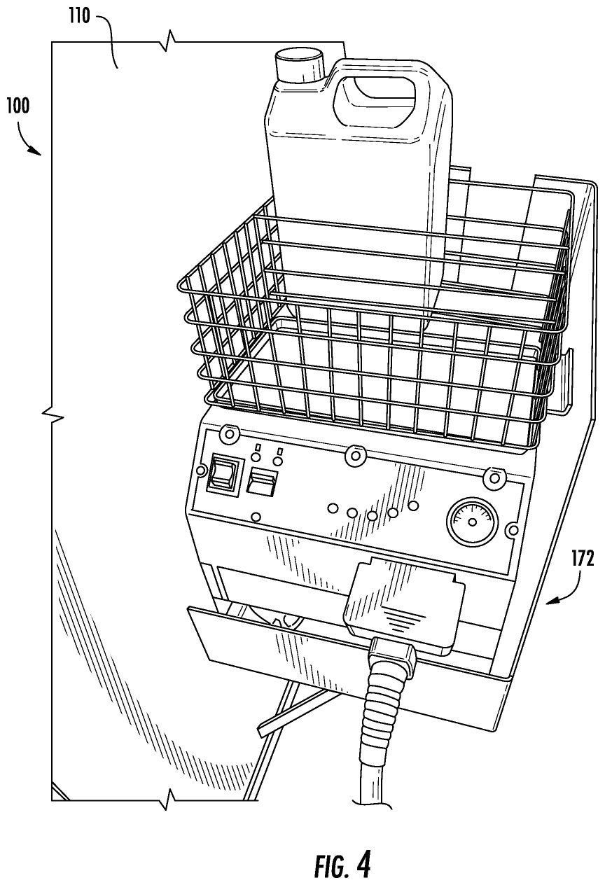

The steam generator 172 shown in FIG. 4 is a 220 VAC VaporJet 8000 from Advanced Vapor Technologies, but in other implementations, the steam generator is any other make or model steam generator capable of producing enough steam to adequately sanitize equipment for a given size processing chamber. The steam generator 172 is held by a stainless-steel shelf, which is supported by two brackets. The gap in the shelf seen in the back is a passageway for the power cord from the rear of the steam generator 172 to the control chassis 190. The steam generator 172 is wired to be powered on via the power switch 144 on the front control panel 142 of the steam cabinet 100, as shown in FIG. 6. The steam generator includes a reservoir for containing water and a float assembly inside the reservoir to determine when to add water to the reservoir. In some implementations, the steam generator includes a “No Water” alarm to indicate that no water is present in the reservoir. In some implementations, the steam generator includes a “Fill Reservoir” alarm to indicate that the water level is low in the reservoir. In some implementations, the steam generator

The steam generator 172 is configured to deliver an agent for cleaning, disinfecting, sanitizing, deodorizing, or any combination thereof. In some implementations, Steam-N-Shield or another agent is included in the steam to assist in cleaning, disinfecting, sanitizing and/or deodorizing the equipment. Cleaning agents, fragrances, antimicrobials can also be used. In a preferred aspect, an antimicrobial nitrogen-containing polysaccharide can be used. In one aspect, chitin is used. In a more preferred aspect, chitosan solution can be used. Such solutions are disclosed in U.S. Pat. No. 9,149,036, which is incorporated by reference herein for its teachings of methods and compositions for sanitizing or disinfecting surfaces.

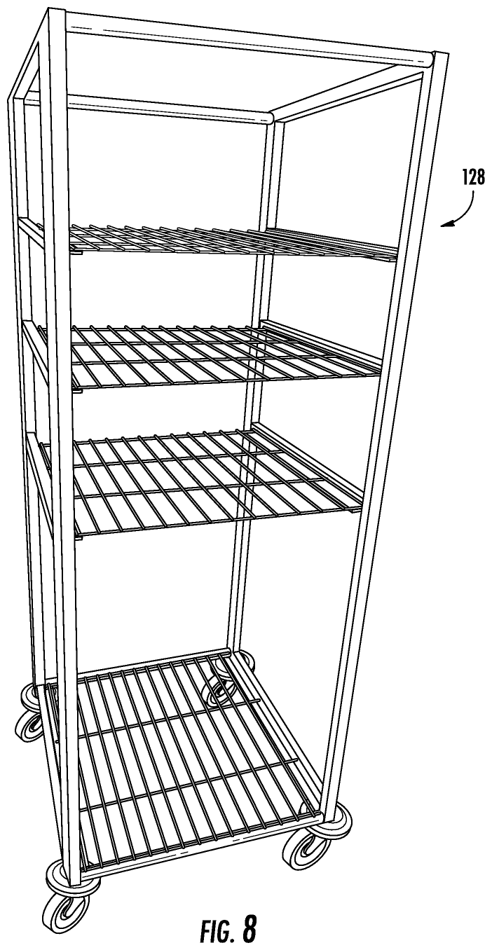

An exhaust fan 180 is included in the steam cabinet 100 to remove the large volume of steam that accumulates inside the processing chamber 102 during the sanitization cycle. This hot steam could pose a safety concern to the operator of the steam cabinet 100 if not vented properly. The exhaust fan 180 has an exhaust fan inlet 182 and an exhaust fan outlet 184. The exhaust fan inlet 182 is ducted to an exhaust port 186 defined by the top chamber wall 114 and is in fluid communication with the processing chamber 102. The exhaust fan outlet 184 can be ducted to a safe location, as shown in FIG. 9. After a sanitation cycle and before the magnetic locking mechanism 122 is released and the items disposed within the processing chamber 102 are removed, the steam should be vented out of the processing chamber 102 using the exhaust fan 180. In some implementations, the steam cabinet includes a pressure relief valve for releasing steam from within the processing chamber.

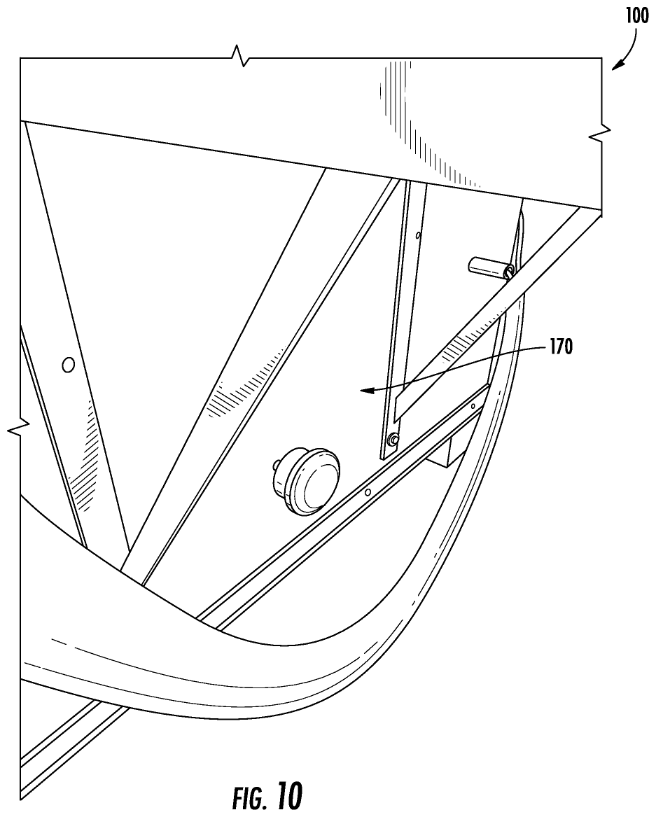

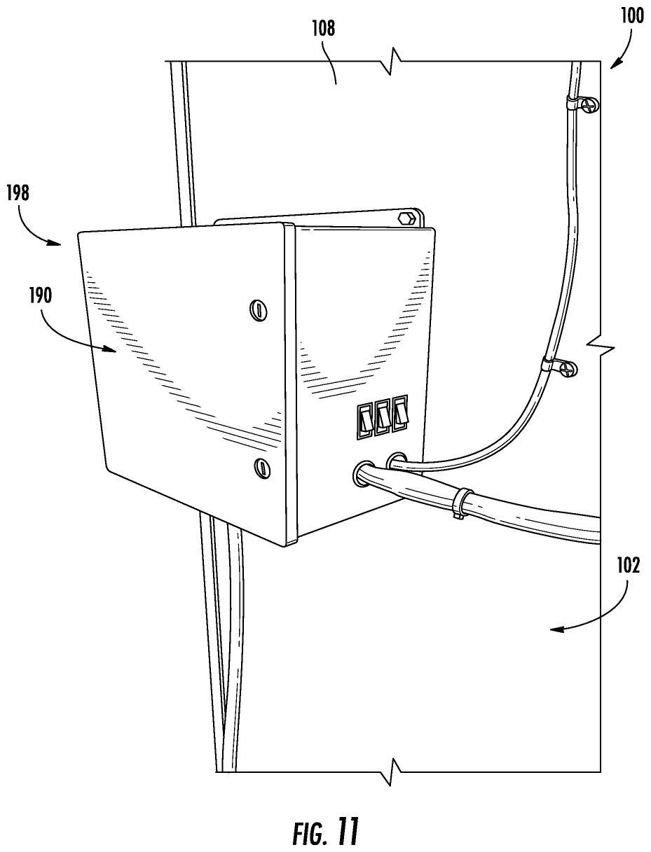

A control chassis 190 is shown in FIG. 11. The control chassis 190 houses the Arduino controller 192 and all associated circuitry for the steam cabinet 100. FIG. 12 shows the interior of the control chassis 190.

In some implementations, the control chassis includes a PLC (Programmable Logic Controller) system to provide system control. The PLC enables adjustments to the sequence and duration of any and all operational functions including: preheat, steam injection, ventilation, and processing chamber locking. The nature of the PLC system is such that it is able to operate independently once programmed. The PLC is capable of monitoring system variables, including temperature and other sensors such as door switches, pressures, and time. In some implementations, the control chassis includes an Arduino controller (or any other make and model controller) and a PLC.

The power system 194 of the disclosed steam cabinet 100 is designed to support rapid heating times. The power system 194 is mounted to the rear of the cabinet. The power system 194 accepts 110 VAC or 220 VAC, preferably 220 VAC.

The power system 194 includes a transformer 196 to power the supply fan 162 and the exhaust fan 180. As shown in FIG. 11, the transformer 194 is mounted to the outside of the control chassis 190 to allow adequate ventilation and cooling. The power system 194 also controls the magnetic door lock 122 for operator safety and the emergency stop button 132 on the interior of the processing chamber 102.

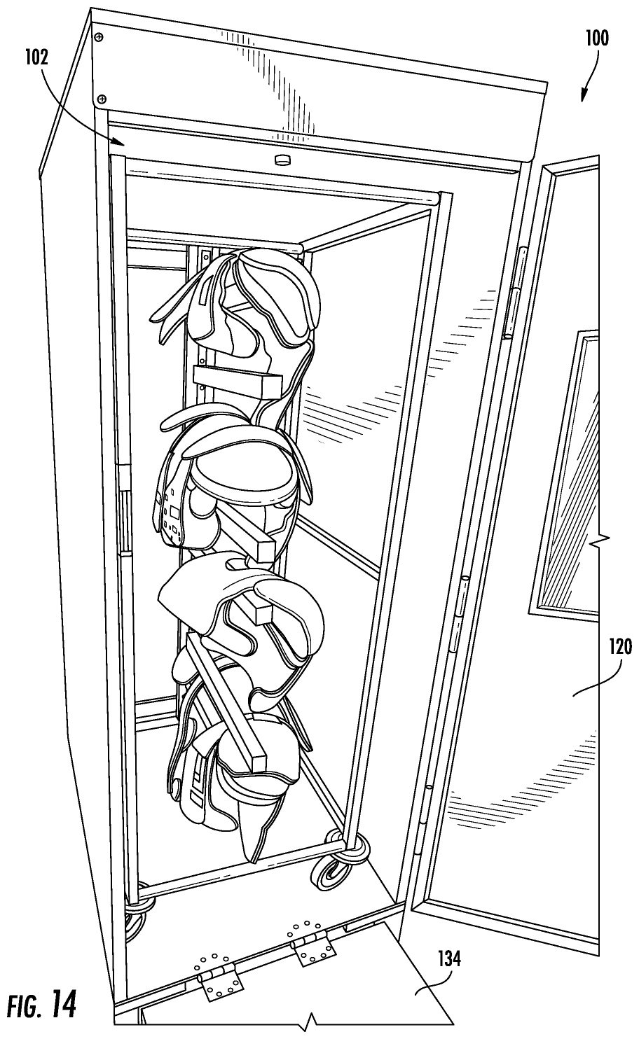

A small 12 VDC power supply 198 is included inside the control chassis 190 to support the Arduino controller 192 and the magnetic door lock 122. The power supply 198 is controlled by the power switch 144 located on the front control panel 142, which acts as the main switch for the entire system. While the power supply 198 is off, no other component receives power. As such, the interior emergency stop button 132 is wired in line with the main power switch 144 to ensure safe operation. For a complete system circuit diagram, see FIG. 16.

There are four systems that can effectively control the sanitation process: (1) the heating element 160, (2) the steam generator 172, (3) the exhaust fan 180, and (4) the magnetic locking device 122. For a “one-button system” and the need for higher heating capability two additional circuits are required: (5) the steam generator power control, and (6) the supply fan control. In total, six systems are used. The Arduino controller 192 is used to sequence the process and was chosen for its ease of use, availability, and low cost.

In use, the sanitation process can be broken down into 3 simple steps: (1) Insert items to be cleaned into the processing chamber 102 and press the appropriate start button 148, 150 for batch size, (2) activate the heating element 160 and steam generator 172 to bring equipment up to predetermined temperature (preferably to 167° F. minimum, as per FDA guidelines) for a predetermined period of time according to the start temperature and batch size, and (3) when the cycle finishes, use the exhaust fan 180 to ventilate the processing chamber 102.

Each step requires certain elements to be powered on, and others to be powered off. For example, during a preheat phase, the heating element 160, supply fan 162, steam generator 172, and magnetic locking device 122 should all receive power, while the exhaust fan circuit 180 should not. This behavior is controlled using the digital input/output (I/O) capabilities of the Arduino controller 192. The timing of process events is sequenced using the internal clock on the Arduino controller 192 board. Each event is user-programmable, so that each step lasts for the appropriate amount of time as determined from the extensive testing.

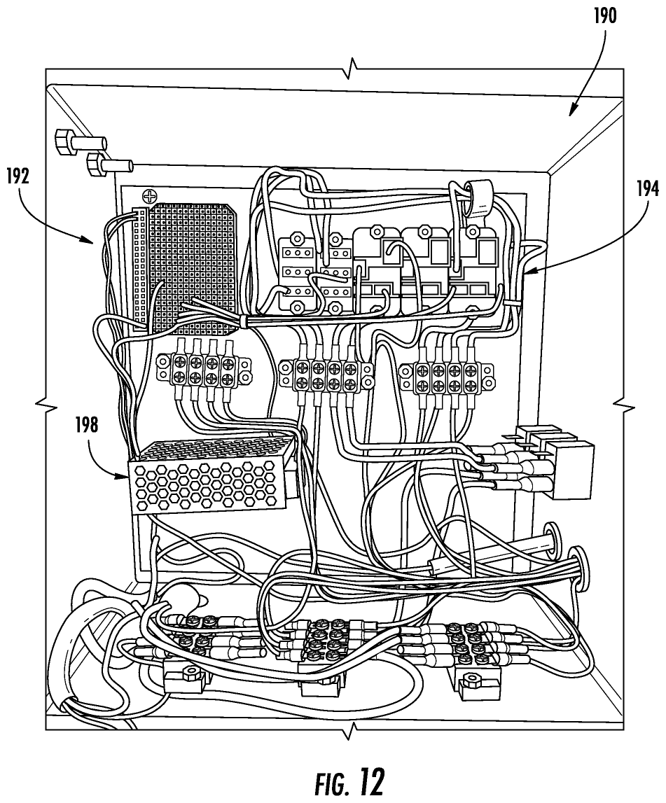

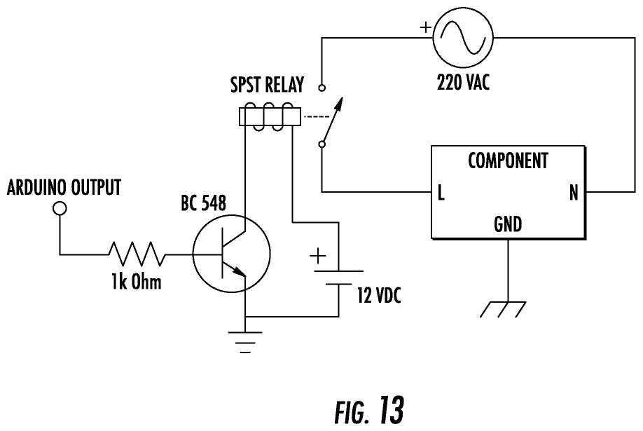

Because the Arduino controller 192 cannot directly control the large currents and voltages required by the system components, a two-step circuit was used, as shown in FIG. 13.

The Arduino controller 192 sends a signal to an NPN transistor when a circuit is to be turned on. The transistor is made conductive by this signal and allows current to flow through the coils of the corresponding relay, which closes the contacts and powers on the appropriate component. The heating element 160, steam generator 172, supply fan 162, exhaust fan 180, and magnetic locking device 122 are all controlled in this manner. The Arduino controller 192 can control injection time by sending a signal to the NPN transistor, which closes and completes the circuit.

Additionally, a temperature sensor 199 (a thermistor) is disposed in the interior of the processing chamber 102 to allow the Arduino controller 192 to measure the temperature inside the processing chamber 102. Using the temperature sensor 199 and another resistor to form a voltage divider, the Arduino controller 192 measures the change in resistance of the thermistor due to temperature as a change in voltage. This allows steam cabinet 100 parameters to be changed based on the temperature inside the processing chamber 102.

### Examples

A vacuum oven was used to test the impact that several parameters had on the absorption of Steam-N-Shield during batch processing. In this testing, steam was injected into the oven chamber containing a small test sample of shoulder pad material. Using a micro-scale, these pieces were weighed before and after steam injection. An increase in the mass of the test piece indicates absorption of the steam, and therefore absorption of the Steam-N-Shield in this environment. The test parameters were time of steam spray, cabinet temperature, steam pressure, and vacuum chamber pressure. To test for the effects of each parameter, the measured test parameter was varied while the other parameters were held constant.

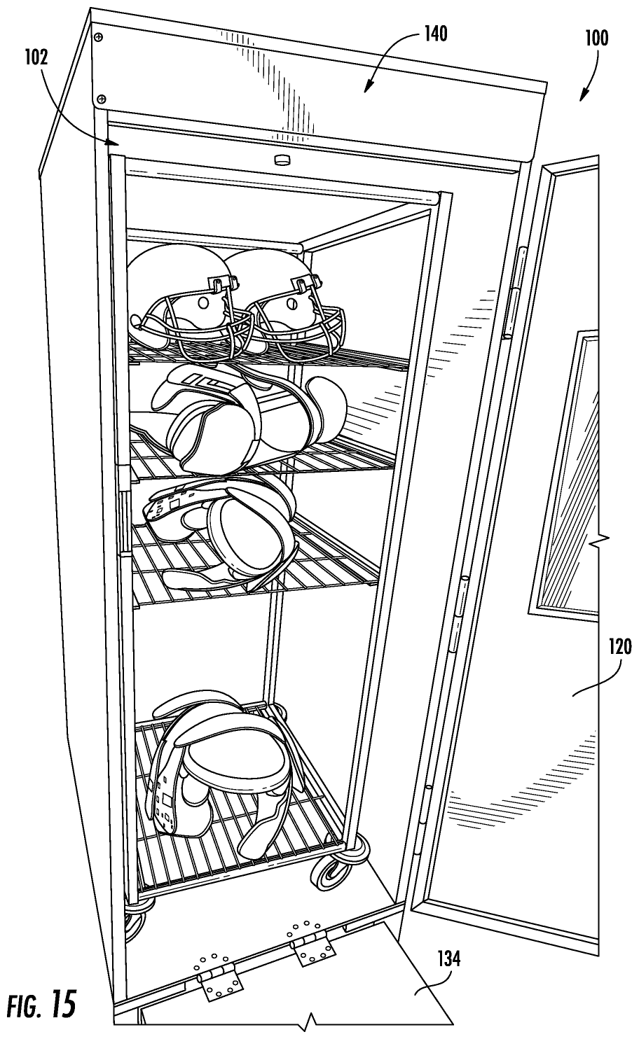

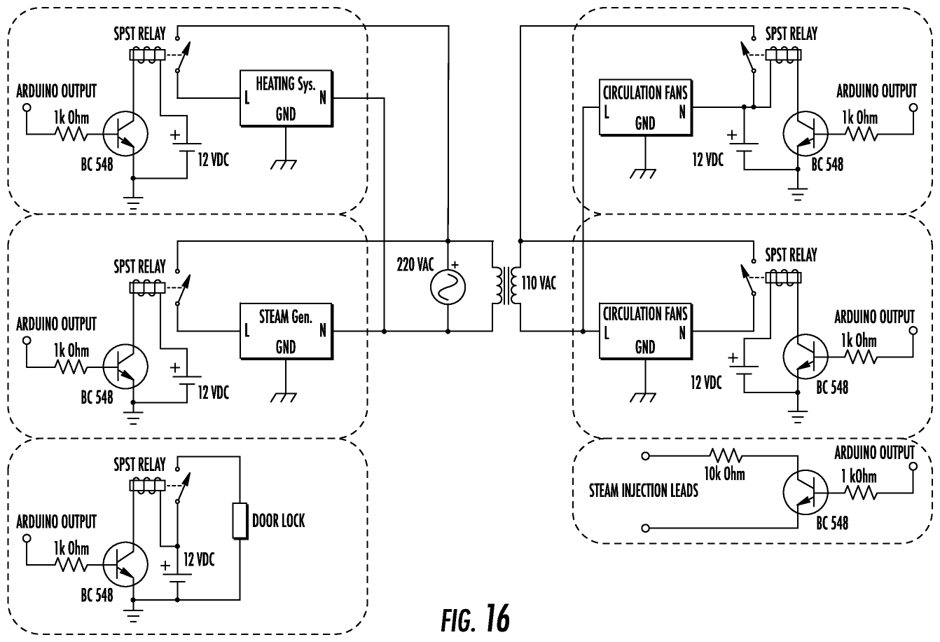

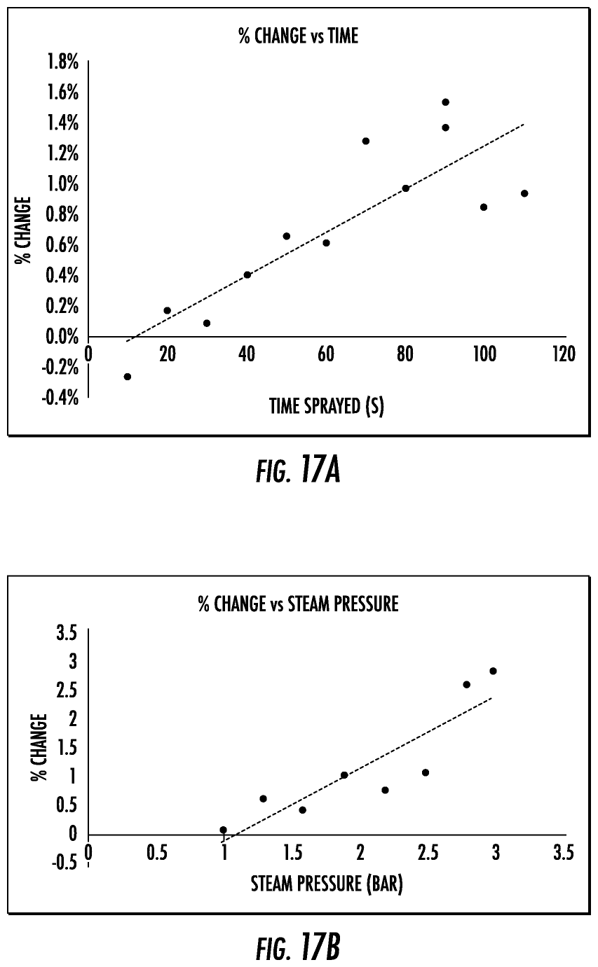

These tests showed that for the best possible absorption of steam, the steam should be injected into the cabinet for as long as possible, at the highest possible steam pressure, and with a cabinet temperature of 160-170 degrees Fahrenheit. Additionally, these tests determined that the vacuum pressure had negligible effect on the amount of steam absorbed by the pads. FIGS. 17A-17D detail the effect that each parameter had on the absorption of the steam.

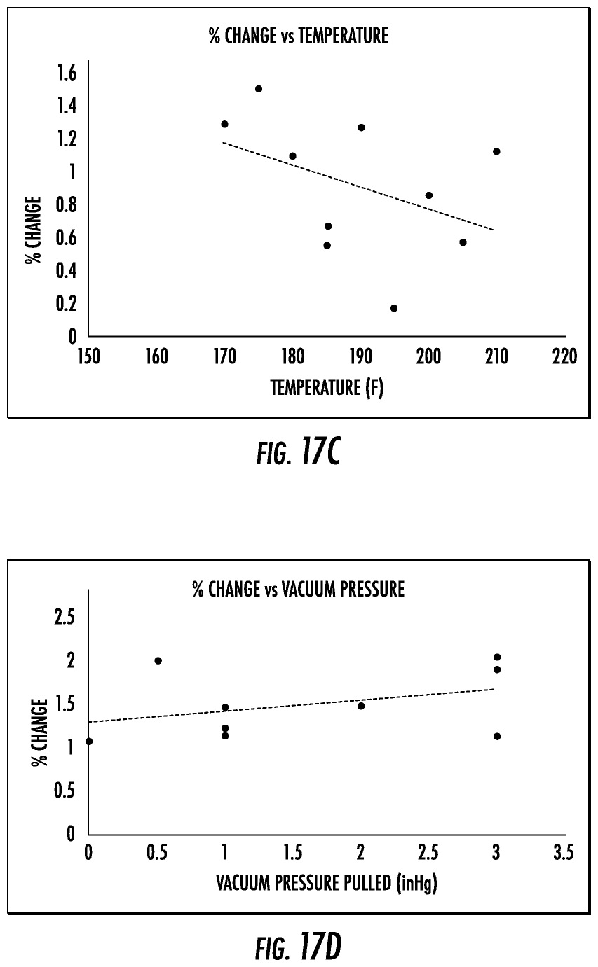

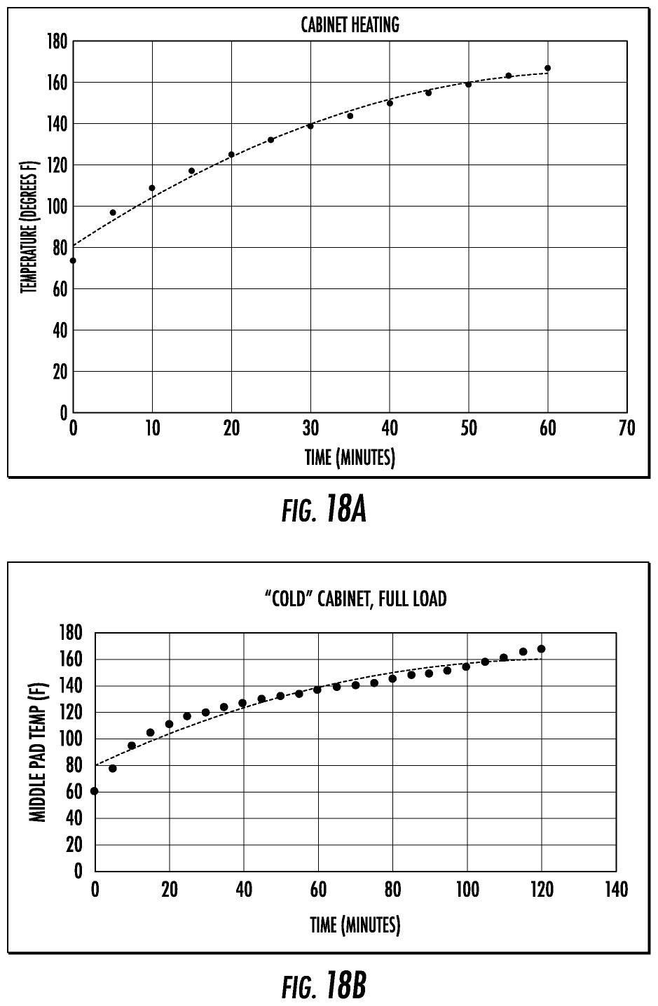

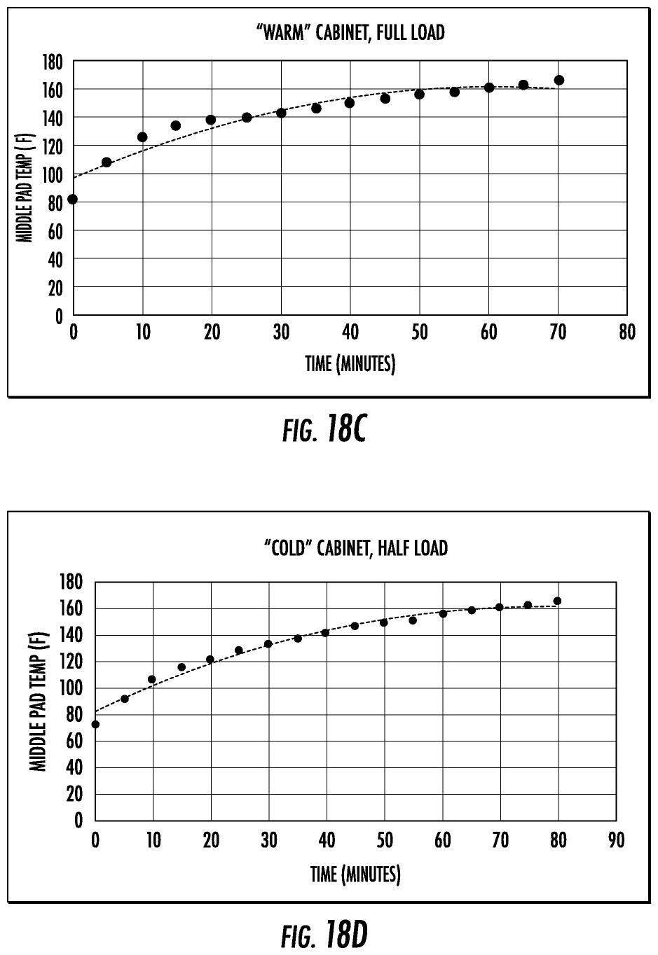

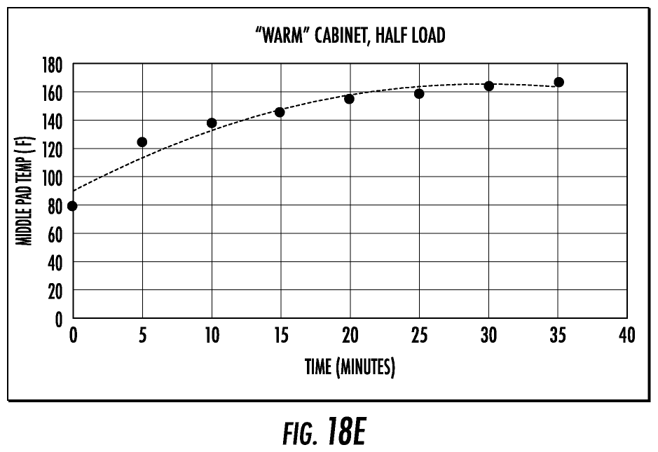

The cabinet takes approximately sixty minutes to heat from ambient temperature to the required 167 degrees Fahrenheit, though it is desired to let the cabinet heat to about 175 degrees before pad insertion, as the cabinet drops a few degrees when the door is opened to load racks. Without any steam injection, the maximum temperature observed inside the cabinet is 196 degrees Fahrenheit, after letting the cabinet heat for three hours. A graph of cabinet heating is included in FIGS. 18A-18E.

In order to expedite the sanitization process, steam can be injected immediately after a rack of equipment is placed in the cabinet, upon the press of one of the cycle start buttons. The injected steam greatly helps in heating the equipment up to the required temperature (167 degrees Fahrenheit), all the while exposing the equipment to the Steam-N-Shield disinfectant. Therefore, by the time the equipment reaches this temperature, it has been absorbing Steam-N-Shield for well over the required batch time of five minutes. The mass flow rate of steam has been calculated as 0.00081 kg/s, and the volumetric flow rate as 0.00081 L/s. These properties were found by measuring the mass of the storage tank (seen on top of the steam generator in the FIG. 4) before and after 800 seconds of steam injection, then dividing the difference in mass by 800 seconds. Since 1 kg of water at standard conditions occupies 1 Liter in volume, it is assumed that these flow rates are equal in magnitude.

A number of tests were conducted to determine the optimal batch process parameters. Steam is injected for the entirety of a cycle, as this allows for the fastest pad heating time. By choosing the appropriate button on the control panel, the user is able to specify whether the cabinet is fully loaded (based on 8 pads in the cabinet), or half loaded (based on 4 pads in the cabinet). A thermistor is used to detect the air temperature within the cabinet, and if the temperature is below 130 degrees Fahrenheit, a “cold” cycle will begin. Otherwise, a “warm” cycle will begin, where the electric heaters are turned off for the first 5 minutes of the cycle to allow for greater condensation buildup on the pads. Data collected to determine these heating times is shown in FIGS. 18B-18E. The time and Steam-N-Shield solution consumption for each cycle is shown below in Table 1. Four sensor probes where used in the cabinet during the experimental setup used for data collection. One sensor probe measured air temperature, and three sensor probes were used to measure the temperature of the middle, highest, and lowest pad, respectively.

The description and properties of each cycle type are as follows:

TABLE 1

| | | | | Solution |
|---|---|---|---|---|
| Cycle Type | Cycle Characteristics | Cycle Length | | Used (L) |
| A | Cold start, full load | 120 | min | 5.83 |
| B | Warm start, full load | 90 | min | 3.89 |
| C | Cold start, half load | 70 | min | 3.40 |
| D | Warm start, half load | 35 | min | 1.71 |

As a final test, the cabinet was loaded with three sets of shoulder pads, and four helmets to simulate a more realistic cycle (one extra helmet to account for the extra thermal mass that would be taken up by cleats, rib pads, etc.). The full cycles were then modified to reflect the results of these tests from a “cold” and “warm” cabinet, with parameters given above. Data collected to determine these heating times is shown in FIGS. 18B-18E. The experimental setup is shown in FIG. 15, where three probes can be seen going into the cabinet. One measured air temperature, one measured the temperature of a set of shoulder pads, and one measured the temperature of a helmet. Throughout the testing procedure, a Weber iGrill temperature sensor was used to track the temperatures of the air inside the cabinet, and the internal temperature of pads in real time. This sensor is accurate within 1 degree Fahrenheit.

The following is a description of a complete sanitization process: (i) When the power is turned on, turn on the steam generator, cabinet heaters, supply fan, and magnetic locking device; (ii) If the door unlock button is pressed at any point, release door for 5 seconds; (iii) When one of two cycle start buttons is pressed, read in the cabinet temperature from the thermistor; (iv) Immediately begin to continuously inject steam when a start button is pressed—if cabinet temperature is below 130 F, turn off heating elements for five minutes; (v) If heating elements were turned off, turn on after five minutes; (vi) After specified cycle time, turn off steam injection and turn on exhaust fan; and (vii) Turn off exhaust fan after specified time and wait for next button press.

For systems that include “Fill Reservoir” and “No Water” alarms, the following is a description of a sanitization process: (i) When entire system is turned on the steamer will also be energized but the heater will not be on; (ii) The controller will check for ‘Fill Reservoir’ alarm—if ON then the system will not be allowed to proceeded until alarm is OFF; (iii) Next the controller should check for ‘No Water’ alarm—if ON then the system cannot proceeded until alarm is OFF; (iv) Once controller has satisfied both of the above conditions then the controller can send a signal to turn the boiler heater on; (v) Once the heater is on the controller will do a hold off time of 15 minutes for 300 F, or 20 Minutes for 340 F, or 30 minutes for 380 F; (vi) When the hold off time is reached the controller can then turn the solenoid on to release steam into the main cleaning chamber for whatever length of time is necessary.

During operation conditions, the controller needs to monitor and/or control: (i) If ‘No Water’ alarm comes on after initial startup then the heater must be turn OFF and the cycle must stop as this will be a major problem that needs to be corrected—otherwise permanent damage may occur to heater and other equipment; and (ii) If ‘Fill Reservoir’ alarm comes on after initial startup the system can continue normal operation until the cycle is complete—there should be enough solution left in the boiler to finish the cycle without shutting it down.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A steam cabinet comprising:
at least one chamber wall defining a processing chamber;
a door for allowing access to the processing chamber;
at least one heating element configured to provide heat to the processing chamber;
at least one steam generator configured to provide steam to the processing chamber;
a supply fan having a supply fan inlet and a supply fan outlet, wherein the supply fan outlet is in fluid communication with the at least one heating element and the processing chamber; and
an exhaust fan having an exhaust fan inlet and an exhaust fan outlet, wherein the exhaust fan inlet is in fluid communication with the processing chamber.

2. The steam cabinet of claim 1, wherein the at least one chamber wall comprises a first chamber wall, a second chamber wall, a third chamber wall, a fourth chamber wall, a bottom chamber wall, and a top chamber wall, wherein the first chamber wall defines a door opening, and wherein the door is configured to seal the door opening when the door is in a closed position.

3. The steam cabinet of claim 1, wherein the at least one chamber wall comprises insulation.

4. The steam cabinet of claim 2, wherein a bottom portion of the fourth chamber wall defines a steam injection port for providing steam from the at least one steam generator to the processing chamber.

5. The steam cabinet of claim 1, comprising an air filter in fluid communication with the supply fan outlet.

6. The steam cabinet of claim 5, wherein the air filter is a HEPA air filter.

7. The steam cabinet of claim 1, wherein the at least one steam generator is configured to deliver an agent for cleaning, disinfecting, sanitizing, deodorizing, or any combination thereof.

8. The steam cabinet of claim 7, wherein the agent comprises an antimicrobial agent.

9. The steam cabinet of claim 1, wherein the at least one heating element and the at least one steam generator are configured to increase a temperature of the processing chamber to 167 degrees F. or more.

10. The steam cabinet of claim 1, wherein the door comprises a magnetic locking mechanism.

11. The steam cabinet of claim 1, comprising a temperature sensor disposed within the processing chamber.

12. A method of using a steam cabinet, the method comprising:
providing a steam cabinet comprising:
at least one chamber wall defining a processing chamber,
a door for allowing access to the processing chamber,
at least one heating element configured to provide heat to the processing chamber,
at least one steam generator configured to provide steam to the processing chamber,
a supply fan having a supply fan inlet and a supply fan outlet, wherein the supply fan outlet is in fluid communication with the at least one heating element and the processing chamber, and
an exhaust fan having an exhaust fan inlet and an exhaust fan outlet, wherein the exhaust fan inlet is in fluid communication with the processing chamber;
disposing an item to be steamed within the processing chamber;
activating the at least one heating element, the supply fan, and the at least one steam generator to increase a temperature of the processing chamber to 167 degrees F. or more for a predetermined period of time; and
activating the exhaust fan to remove excess steam from the processing chamber.

13. The method of claim 12, wherein the at least one chamber wall comprises a first chamber wall, a second chamber wall, a third chamber wall, a fourth chamber wall, a bottom chamber wall, and a top chamber wall, wherein the first chamber wall defines a door opening, and wherein the door is configured to seal the door opening when the door is in a closed position.

14. The method of claim 12, wherein the at least one chamber wall comprises insulation.

15. The method of claim 13, wherein a bottom portion of the fourth chamber wall defines a steam injection port for providing steam from the at least one steam generator to the processing chamber.

16. The method of claim 12, comprising an air filter in fluid communication with the supply fan outlet.

17. The method of claim 16, wherein the air filter is a HEPA air filter.

18. The method of claim 12, wherein the at least one steam generator is configured to deliver an agent for cleaning, disinfecting, sanitizing, deodorizing, or any combination thereof.

19. The method of claim 18, wherein the agent comprises an antimicrobial agent.

20. The method of claim 12, wherein the door comprises a magnetic locking mechanism.

21. The method of claim 12, comprising a temperature sensor disposed within the processing chamber.
